(19) United States
(12) Patent Application Publication
(10) Pub. No.: US 2020/0206144 A1
(43) Pub. Date: Jul. 2, 2020

(54) Targeted Nanoparticles for Glioblastoma Theranostics

(71) Applicant: Lauren Lukas VanderSpek, Glendale, CA (US)

(72) Inventor: Lauren Lukas VanderSpek, Glendale, CA (US)

(21) Appl. No.: 16/238,401

(22) Filed: Jan. 2, 2019

(51) Int. Cl. A61K 9/51; A61K 9/00; A61K 47/34; A61K 31/5377; A61K 49/18; A61K 49/04

(52) U.S. Cl. CPC A61K 9/5115; A61K 9/0053; A61K 47/34; A61K 9/5146; A61K 31/5377; A61K 49/1818; A61K 49/04; A61K 9/5123

(57) ABSTRACT

Targeted nanoparticles are provided which facilitate detection of and therapy for glioblastoma multiforme (GBM). The nanoparticles may be used to target other forms of cancer as well, such as pancreatic, colorectal, and breast. The nanoparticles may provide optical contrast for pre-operative diagnostic imaging and intraoperative navigation using surface-enhanced Raman scattering techniques. Moreover, the nanoparticles may inhibit tumoral growth, block tumoral blood flow, and decrease metastatic spread of GBM. The nanoparticles may further reduce the inflammatory response, which is essential to the growth of the glioma and can be harmful to the patient. The nanoparticle may comprise a biologically inert substance, a biocompatible polymer, an optical-acoustic reporter, and a glioblastoma specific receptor ligand conjugated to the biocompatible polymer. For instance, in some embodiments, the biologically inert substance may be a gold or silica nanoshell, the biocompatible polymer may be polyethylene glycol, the optical-acoustic reporter may be prussian blue, and the glioblastoma specific receptor ligand may be aprepitant.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to nanoparticles and, more particularly, to targeted nanoparticles for detection of and therapy for glioblastoma multiforme.

### BACKGROUND

Glioblastoma multiforme (GBM) is a highly malignant brain tumor known for its aggressiveness and invasiveness. The outer cell membrane of GBM has certain G-protein coupled receptors, including neurokinin-1 (NK1), which mediate intracellular signaling pathways, leading to an increase in DNA synthesis and cellular proliferation. Glioma cells overexpress NK1 receptors (NK1R) as compared to normal cells. Further, the degree of malignancy of a tumor is positively correlated with the number of NK1R.

NK1R has two endogenous peptide agonists, substance P (SP) and hemokinin-1 (HK-1). HK-1 favors the migration of glioma cells and exerts this action via the NK1R. SP, on the other hand, is the preferential endogenous ligand for NK1R and regulates proliferation and migration of glioma cells. Indeed, the NK1R/SP system stimulates angiogenesis and inflammation, thereby contributing to glioma progression. More particularly, in inflammation, the NK1R/SP system is upregulated and glioma cells overexpress NK1R. To halt growth of GBM, it is necessary to block this inflammatory response.

Necrosis, a severe hypoxia, is a histological hallmark of GBM and a predictor of poor prognosis. Under hypoxic conditions, SP upregulates the expression of hypoxia inducible factor (HIF), which controls the tumor response. Moreover, under normal conditions, hypoxia facilitates the self-renewal of normal stem cells. In glioma, hypoxia promotes stem cell-like phenotypes.

Characterized as a grade IV tumor by the World Health Organization, the median survival time of patients after treatment is approximately 13 months. GBM develops from star-shaped glial cells, known as astrocytes, that support the health of the brain's neurons. The boundaries of GBM are typically not well-defined due to its finger-like tentacles that aggressively infiltrate normal brain tissue. For this reason, complete removal of GBM can be challenging, especially at functional regions of the brain, and recurrence is common. However, the ability to precisely locate and distinguish GBM cells from normal brain tissue is crucial for effective treatment.

Currently, there is no cure for GBM. Most treatments focus on surgical resection of the tumor. This method remains problematic because any invasive procedure causes “surgical injury” to the brain through initiation of the inflammatory response and angiogenesis. These natural responses, in turn, facilitate growth of the glioma. Indeed, in 90% of patients, the recurrence of glioma occurs in the tumor resection margin after surgery.

Another issue with surgical removal of GBM is lack of ability to reliably define the tumor's borders for excision. In particular, ultrasound, positron emission tomography (PET), magnetic resonance imaging (MRI), and computed tomography (CT) have been extensively used but still have serious drawbacks. For instance, PET requires the application of radioactive tracers, which can be harmful to the patient. Additionally, spatial resolution and patient scanning and transport time inhibit accurate imaging. Moreover, assessment of tumor borders by preoperative imaging is often incongruent with the tumor's actual borders during surgery due to brain shift during the operative procedure.

One proposed solution involves utilization of an imaging agent, such as aminolevulinic acid hydrochloride (ALA HCl), for fluorescence guided surgery. The ALA HCl is injected and metabolized so as to accumulate in GBM cells, producing fluorescence of those cells when illuminated by a special blue light. Fluorescent labels are problematic due to their fragile nature and consequent reduced contrast from organelles or other components of the tissue. Further, large amounts of fluorophores may become toxic to the patient.

There remains a need for compositions useful in the diagnosis and treatment of cancer, and more specifically, glioblastoma multiforme.

### SUMMARY

The present disclosure is directed to nanoparticles that facilitate detection of and therapy for glioblastoma multiforme. More particularly, the nanoparticles may provide optical contrast for pre-operative diagnostic imaging and intraoperative navigation, as well as, reduce edema and inhibit GBM growth. The nanoparticles may be used to target other forms of cancer as well, such as pancreatic, colorectal, and breast.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the nanoparticle may comprise a biologically inert substance, a biocompatible polymer, an optical-acoustic reporter, and a glioblastoma specific receptor ligand. The biologically inert substance may be a nanoshell, which may comprise gold or silica. The gold or silica nanoshell may increase the effective dose of radiation to GBM and decrease radiation doses to normal, non-diseased brain tissue. Moreover, the nanoshell may act as a contrast agent for diagnostic imaging, such as MRI and CT. In other embodiments, the biologically inert substance may comprise another shape such as a nanorod, a nanostar, or a nanocube. A person of ordinary skill in the art will recognize that the biologically inert substance may comprise other shapes and structures as well.

According to certain embodiments, the glioblastoma specific receptor ligand may be conjugated to the biocompatible polymer. In some embodiments, the biocompatible polymer may be polyethylene glycol (PEG). The polyethylene glycol may reduce accumulation of the nanoparticle in the reticuloendothelial system, increase circulation time, and decrease the extent to which the nanoparticle binds to non-targeted tissues. One of ordinary skill in the art will recognize other biocompatible polymers may be used in accordance with this invention. For instance, the biocompatible polymer may comprise polycarbonates, polyanhydrides, or polyhydroxyacids. In other embodiments, the biocompatible polymer may comprise a combination of known polymers.

In some embodiments, the optical-acoustic reporter may be prussian blue. Prussian blue may provide a surface-enhanced Raman scattering (SERS) tag. More particularly, the nanoparticles may target the glioblastoma cells so as to amplify Raman signals from those cells. Other optical-acoustic reporters may be utilized in accordance with this invention, such as gadolinium chelates, superparamagnetic iron oxide, and hematoxylin-eosin.

In certain embodiments, the glioblastoma specific receptor ligand may be a neurokinin-1 receptor (NK1R) antagonist. In some embodiments, the NK1R antagonist may be aprepitant. Aprepitant may target the NK1R in GBM and peritumoral vessels. In this way, aprepitant may allow visualization of GBM for diagnostic imaging. Aprepitant may also inhibit GBM growth, block tumoral blood flow, and decrease metastatic spread. Further, aprepitant may also have anti-emetic properties, thereby reducing nausea and vomiting normally associated with cancer chemotherapy.

One or more of the above-disclosed embodiments, in addition to certain alternatives, are provided in further detail below. The disclosed subject matter is not, however, limited to any particular embodiment disclosed.

### ADVANTAGES

Several advantages of one or more aspects are to provide targeted nanoparticles for detection of and therapy for glioblastoma multiforme that:
(a) specifically targets neurokinin-1 receptors, which are highly expressed by glioblastoma cells;
(b) provides highly sensitive detection of the boundaries of a tumor pre-operatively and intraoperatively;
(c) inhibits GBM growth;
(d) reduces the inflammatory response;
(e) blocks tumoral blood flow;
(f) decreases metastatic spread;
(g) prolongs the circulation half-life in vivo;
(h) increases the effective dose of radiation;
(i) has a safe toxicity profile;
(j) can be orally delivered; and
(k) has anti-emetic properties.

These and other advantages of one or more aspects will become apparent from consideration of the ensuing description. Although the description above contains many specifics, these should not be construed as limiting the scope of the embodiments but as merely providing illustrations of some of several embodiments. Thus, the scope of the embodiments should be determined by the claims that are appended and their legal equivalents, rather than by the examples given. The description of the invention which follows should not be construed as limiting the invention to the examples shown and described, because those skilled in the art to which this invention pertains will be able to devise other forms thereof within the ambit of the appended claims.

### DETAILED DESCRIPTION

Having summarized various aspects of the present disclosure, reference will now be made in detail to various embodiments of the present invention. While the disclosure will comprise specific details, there is no intent to limit it to the embodiment or embodiments disclosed herein. Rather, the intent is to cover all alternatives, modifications and equivalents included within the spirit and scope of the disclosure as defined by the appended claims.

Briefly described, one embodiment, among others, is a nanoparticle that comprises a biologically inert substance, a biocompatible polymer, an optical-acoustic reporter, and a glioblastoma specific receptor ligand. The glioblastoma specific receptor ligand may be conjugated to the biocompatible polymer. The nanoparticle may be administered orally. In other embodiments, the nanoparticle may be intravenously delivered.

In certain embodiments, the biologically inert substance may comprise a nanoshell. Further, the nanoshell may be formed of gold or silica. In embodiments where the nanoshell comprises gold, the gold may be inert and may be safe for use in biomedical applications. Silica may also be inert. In some embodiments, the nanoshell may be spherical in shape and consist of a dielectric core covered by a thin metallic shell. Also, the nanoshell may be characterized by a low toxicity profile and therefore, may be safe for use in humans. Moreover, the nanoshell may provide flexibility as to which molecules may be conjugated thereto.

In other embodiments, the biologically inert substance may comprise other shapes as well. For instance, the biologically inert substance may comprise a nanorod, a nanostar, a nanotube, a nanocube, a nanosphere, a nanocapsular particle, or other nanomaterials. Indeed, a person of ordinary skill in the art will recognize that the biologically inert substance may comprise numerous other structures.

The nanoshell may be transported across the blood-brain barrier, thereby permitting therapeutic drugs to be delivered to diseased areas of the brain. The nanoshell may further act as a contrast agent for diagnostic imaging. More particularly, the nanoshell may allow for greater visualization of GBM for diagnostic MRI and CT. Additionally, the nanoshell may increase the Raman scattering signal using SERS, which, in turn, may permit highly sensitive detection of microscopic portions of a tumor. In application, intraoperative SERS probes, scanners, or other implements may be used to detect the boundaries of a tumor during surgical resection.

Moreover, the nanoshell may increase the effective dose of radiation applied to a tumor, peritumoral stroma, and peritumoral vessels. During radiotherapy, the nanoshell may cause radiation from within the tumor. Thus, the nanoshell may improve the therapeutic window by allowing lower radiation doses to normal brain tissue while increasing the effective dose to diseased cells.

In certain embodiments, the biocompatible polymer may be polyethylene glycol (PEG). PEG may be an inert, coiled polymer comprised of repeating ethylene ether units with dynamic conformations. In other embodiments, the biocompatible polymer may comprise polyalkylene glycol. A person of ordinary skill in the art will appreciate that many other biocompatible polymers may be used in accordance with this invention. For instance, the biocompatible polymer may comprise polyethylene, polyvinyl alcohols, polyhydroxyacids, polycarbonate, polyanhydrides, polyesters, polypropylfumerates, polyamines, polycaprolactones, polyamides, polyacetals, polyethers, polycyanoacrylates, polyurethanes, polyphosphazenes, polyacrylates, polymethacrylates, polyureas, polystyrenes, or combinations thereof.

The biocompatible polymer may prolong the circulation half-life of the nanoparticle in vivo. Indeed, the biocompatible polymer may reduce accumulation of the nanoparticle in the reticuloendothelial system (RES). In this way, the biocompatible polymer may mitigate concerns about nanoparticle toxicity, which often arises due to accumulation in the RES. The biocompatible polymer may also decrease association of the nanoparticle to non-targeted serum and tissue proteins, thereby resulting in “stealth” behavior.

In some embodiments, the optical-acoustic reporter may be prussian blue. Prussian blue may be a cyanide-bridged coordination polymer dye characterized by a strong and sharp single vibrational peak at 2156 cm(−1) throughout the whole Raman spectrum. Prussian blue may provide a SERS tag that may associate with and effectively label diseased tissue. Specifically, prussian blue may facilitate the use of Raman spectroscopy, which may be used for intraoperative differentiation of brain tumor from normal brain tissue. Use of prussian blue and Raman spectroscopy may be nondestructive, non-invasive, and may provide information about the molecular composition and structure of GBM. Through this application, the optical-acoustic reporter may provide high sensitivity and specificity of detection and may also reduce false positive signals, leading to less resection of normal brain tissue. Together with the nanoshell, the optical-acoustic reporter may enhance the SERS signal, allowing use of intraoperative SERS probes, scanners, or other implements for neurosurgical intraoperative navigation. One of ordinary skill in the art will recognize that other optical-acoustic reporters may be implemented in accordance with this invention. For instance, the optical-acoustic reporter may comprise galodium chelate, superparamagnetic iron oxide, and hematoxylin-eosin.

As mentioned previously, the glioblastoma specific receptor ligand may be conjugated to the biocompatible polymer. In certain embodiments, the glioblastoma specific receptor ligand may be a neurokinin-1 receptor (NK1R) antagonist. More specifically, the NK1R antagonist may target NK1R in GBM and peritumoral vessels and may competitively inhibit SP and HK-1 binding to the NK1R. This competitive inhibition of the SP/NK1R system may, in turn, block the effects of both SP and HK-1 and may decrease angiogenesis and the inflammatory response mediated by the NK1R. The NK1R antagonist may block the breakdown of glycogen in glioblastoma cells and since cancer cells require a high level of glucose, proliferation of such cells may be inhibited. Additionally, the NK1R antagonist may prevent the migration of glioblastoma cells by blocking changes in cellular shape mediated by SP, including blebbing. The NK1R antagonist may even induce the death of glioblastoma cells by apoptosis.

In other embodiments, the NK1R antagonist may also exert an anti-tumor action against cancer stem cells by inhibiting the pathways associated with hypoxia-mediated maintenance of glioblastoma stem cells. Moreover, due to the high density of NK1R in GBM, the NK1R antagonist may target the NK1R in GBM and peritumoral vessels to facilitate visualization of the tumor for diagnostic imaging. For example, the NK1R antagonist binding to the NK1R in GBM may result in a gradient of enhancement on MRI and a density gradient on CT imaging.

In some embodiments, the NK1R antagonist may be aprepitant. Aprepitant may be lipid soluble and may readily cross the blood-brain barrier, allowing for high concentrations to be reached in the central nervous system. Aprepitant may be well-tolerated and safe for human consumption. Further, aprepitant may treat cancer chemotherapy induced nausea and vomiting.

According to certain embodiments of this invention, the pharmaceutical composition may comprise a plurality of nanoparticles. Each of the plurality of nanoparticles may comprise a biocompatible polymer, an optical-acoustic reporter, and a glioblastoma specific receptor ligand conjugated to the biocompatible polymer.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, the glioblastoma specific receptor ligand may bind other types of receptors other than the NK1R. In this manner, the nanoparticle may target and treat other types of cancer, such as pancreatic, colorectal, or breast cancer. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the targeted nanoparticles for glioblastoma theranostics with which that terminology is associated. In general, the terms used in the following claims should not be constructed to limit the targeted nanoparticles for glioblastoma theranostics to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the targeted nanoparticles for glioblastoma theranostics is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the targeted nanoparticles for glioblastoma theranostics.

1. A nanoparticle comprising:
a biologically inert substance, wherein the biologically inert substance is a nanoshell;
a biocompatible polymer;
an optical-acoustic reporter; and
a glioblastoma specific receptor ligand conjugated to the biocompatible polymer.

2. The nanoparticle of claim 1, wherein the nanoshell is comprised of gold or silica.

3. The nanoparticle of claim 1, wherein the biocompatible polymer is polyethylene glycol.

4. The nanoparticle of claim 1, wherein the optical-acoustic reporter is prussian blue.

5. The nanoparticle of claim 1, wherein the glioblastoma specific receptor ligand is a neurokinin-1 receptor antagonist.

6. The nanoparticle of claim 5, wherein the neurokinin-1 receptor antagonist is aprepitant.

7. A pharmaceutical composition comprising:
a plurality of nanoparticles, wherein each nanoparticle comprises:
a biocompatible polymer;
an optical-acoustic reporter; and
a glioblastoma specific receptor ligand conjugated to the biocompatible polymer.

8. The pharmaceutical composition of claim 7, wherein the composition is administered orally.

9. The pharmaceutical composition of claim 7, wherein the plurality of nanoparticles comprise gold nanoshells.

10. The pharmaceutical composition of claim 7, wherein the plurality of nanoparticles comprise silica nanoshells.

11. The pharmaceutical composition of claim 7, wherein the biocompatible polymer is polyethylene glycol.

12. The pharmaceutical composition of claim 7, wherein the optical-acoustic reporter is prussian blue.

13. The pharmaceutical composition of claim 7, wherein the glioblastoma specific receptor ligand is a neurokinin-1 receptor antagonist.

14. The pharmaceutical composition of claim 13, wherein the neurokinin-1 receptor antagonist is aprepitant.

15. A nanoparticle comprising:
a nanoshell, wherein the nanoshell comprises gold or silica;
polyethylene glycol
an optical-acoustic reporter; and
a glioblastoma specific receptor ligand conjugated to the polyethylene glycol.

16. The nanoparticle of claim 15, wherein the optical-acoustic reporter is prussian blue.

17. The nanoparticle of claim 15, wherein the glioblastoma specific receptor ligand is a neurokinin-1 receptor antagonist.

18. The nanoparticle of claim 17, wherein the neurokinin-1 receptor antagonist is aprepitant.
